(19) United States
(12) Patent Application Publication
(10) Pub. No.: US 2025/0255655 A1
(43) Pub. Date: Aug. 14, 2025

(54) Sacroiliac Fixation Locking Bolt

(71) Applicant: Alesha Scott, Lincoln, NE (US)

(72) Inventor: Alesha Scott, Lincoln, NE (US)

(21) Appl. No.: 19/196,715

(22) Filed: May 1, 2025

(51) Int. Cl. A61B 17/86; A61B 17/68

(52) U.S. Cl. CPC A61B 17/863; A61B 17/864; A61B 17/8695; A61B 2017/681; A61B 2017/8655

(57) ABSTRACT

The invention is a construct consisting of a fully-threaded screw with an attached locking bolt that secures the posterior pelvis in a transiliac transsacral fashion, in the setting of an unstable posterior pelvic ring fracture. One part of the implant is a cannulated screw that has two pitches of different threads, which is placed over a guidewire and secured on the opposite side with a cannulated locking bolt that affixes to the screw. Washers on either side of the implant provide compression, and cancellous threads across the length provide fixation within the sacrum and the iliac bones on either side. This invention provides added strength and stability compared to current fixation methods consisting of screws in isolation.

### REFERENCE TO RELATED APPLICATIONS

Not applicable

### RELATED APPLICATIONS

Not applicable

### CROSS-REFERENCES

Not applicable

### REFERENCE TO RELATED PATENTS

U.S. Pat. No. 10,064,670 B2 September 2018 Mootien
U.S. Pat. No. 8,814,866 B2 August 2014 Vaidya
U.S. Pat. No. 4,454,876A May 1982 Mears
U.S. Pat. No. 11,986,397B2 July 2020 Reiley
US20160157897A1 Vaidya
US201601437671A1 Jimenez
US20160120661A1 Schell
US20160038186A1 Herzog
US20150342753A1 Donner
US20090099610A1 Johnson
US20200093523A1 Sinnott
International Patent AU2015259466 November 2015 Altmann
International Patent CN110678133A May 2018

### STATEMENT OF GOVERNMENT INTEREST

Not applicable

### RIGHTS TO INVENTION UNDER FEDERAL RESEARCH

Not applicable

### FEDERALLY SPONSORED RESEARCH AND DEVELOPMENT

Not applicable

### GOVERNMENT INTEREST

Not applicable

### GOVERNMENT RIGHTS IN THE INVENTION

Not applicable

### FEDERAL FUNDS STATEMENT

Not applicable

### STATEMENT AS TO RIGHTS TO INVENTIONS MADE UNDER FEDERALLY

Not applicable

### SPONSORED RESEARCH AND DEVELOPMENT

Not applicable

### ORIGIN OF THE INVENTION

Not applicable

### FEDERALLY SPONSORED RIGHTS

Not applicable

### STATEMENT OF FEDERALLY SPONSORED RESEARCH

Not applicable

### ACKNOWLEDGMENT OF GOVERNMENT SUPPORT

Not applicable

### DESCRIPTION

A device to be used for fixation of posterior pelvic ring injuries

### BACKGROUND OF THE INVENTION

Orthopedic surgery is a field in which implants are used to fix various injuries of the skeleton and related structures. It is common to use threaded implants to stabilize bony fractures and immobilize those areas that require some rigidity to heal. However, there are problems with existing fixation implants. Accordingly, there is a need in the art for improved implants that address current problems.

### BRIEF STATEMENT OF THE INVENTION

The invention is related to the device and technique for fixation of unstable posterior pelvic ring fractures. This implant is to be used as a percutaneous, minimally-invasive surgical treatment for unstable pelvic fractures utilizing the internal implant that traverses the ilia and sacrum, through a transiliac transsacral bony corridor. This device has been developed to improve bony fixation available in the present state, and in response to the problems that arise with current issues with the available compression fasteners. The invention is placed using established surgical methods of posterior pelvic ring fixation with modifications to account for the improvements made to the implant.

### SUMMARY OF THE INVENTION

A sacral fixation device can consist of a screw implant, a guide wire, a locking component, and washers. The washers provide a surface upon which forces can be more evenly distributed against the iliac bones on either side of a fracture, which requires compression and stable fixation. The washer acts as an abutment that disallows the screw head from perforating the bony cortex and causing injury or losing fixation in the cancellous bone of the posterior pelvis. The guide wire provides a trajectory for fixation and a point of contact via which surgery can be performed in a percutaneous fashion, through small incisions. The dual pitch along the screw length allows a locking bolt to be affixed on the far side of the implant, which maintains its fixation on the opposite side of the pelvis via threads on its outer surface, and which locks to the screw implant via threads on its inner surface.

### OBJECT OF THE INVENTION

One object of the invention is to improve bony fixation in cases of pelvic ring injury requiring surgical treatment. Another object is to improve patient comfort and longevity of fixation with increased stability in the bone. A third object is to reduce or eliminate the need for revision surgery that can arise with fixation failure.

### FIELD OF THE INVENTION

The invention relates generally to methods and devices for the surgical treatment of bone and, more particularly, to the stabilization of bones with an intramedullary device.

### PRIOR ART

See Reference to Related Patents above for prior art

### DESCRIPTION OF THE PRIOR ART

Other sacral and sacroiliac fixation devices, and orthopedic implants.

### BRIEF STATEMENT OF THE PRIOR ART

Internal and external fixation devices for orthopedic injuries.

### BRIEF DESCRIPTION OF DRAWINGS

FIG. 1. shows the sacroiliac fixation device fully assembled.

FIG. 2. is a perspective view of the screw portion of the sacroiliac fixation device with its outer threads of two variable pitches, one along the main length of the implant and one constituting the distal two centimeters.

FIG. 3. is an enlarged perspective view of the distal tip of the screw with the various parts of the invention specifically labeled.

FIG. 4. depicts a perspective view of the locking bolt portion of the sacroiliac fixation device.

FIG. 5. is an exploded perspective view of the sacroiliac fixation locking bolt parts over a guidewire.

FIG. 6. is a depiction of the sacroiliac fixation locking bolt device as it would be assembled in the bony pelvis to secure an injury.

FIG. 7. is a cross-sectional view of a human pelvis showing the sacroiliac locking fixation bolt implant assembled within a safe corridor.

FIG. 8. is an anatomical drawing showing the structures of the human pelvis.

### JOINT RESEARCH AGREEMENT

Not applicable

### BEST MODE FOR CARRYING OUT THE INVENTION

The best mode for carrying out the invention is to employ the device herein to securely fix posterior pelvic ring injuries, placing the implants over a guidewire and securing the locking bolt to the screw with washers on either side to abut the lateral iliac cortices.

### DETAILED DESCRIPTION OF THE INVENTION

The pelvic ring consists of multiple bones that fuse together during skeletal development to form a stiff structure that supports the internal organs and allows the lower appendicular skeleton, the legs, to attach to the axial skeleton, the spine. The iliac bones provide more than two-thirds of the bony structure to the pelvis, and are joined at the back of the pelvis by the sacrum, the tailbone. There is a fibrocartilaginous connection in the anterior pelvis at the region of the pubic symphysis. The sacroiliac joints at the posterior pelvis are secured together with ligaments anteriorly and posteriorly, similar to a suspension bridge. When the pelvis is broken, it is considered unstable if the patient cannot mobilize, or if there is significant displacement as seen on radiographs or computerized tomography (CT). Unstable pelvic fractures may be treated either internally or externally, based on multiple factors including patient hemodynamic stability, fracture pattern, and surgeon training and experience.

Pelvic ring injuries are common, and can occur as a result of a low- or a high-energy trauma, and can involve fractures, ligamentous tears, or a combination of the two. Examples of a low-energy trauma would be a ground-level fall resulting in an elderly person sustaining a pelvic fracture. An example of a high-energy trauma would be a motor vehicle collision, a fall from a great height, or a crush by a large object, leading to an injury to the pelvic ring. Pelvic fractures can be deadly when a patient does not receive surgical treatment in an appropriate timeframe and manner. To ensure the best recovery and return to function, the pelvis must be fixed in a stable way, which can be challenging given the cancellous nature of the bone.

Unstable pelvic fractures pose a unique problem due to the physical nature of the posterior bone, the sacrum, and the current lack of options for fixation of these injuries. The sacral bone is cancellous, which lends to difficulty in obtaining adequate bony purchase with current implants available on the market. Cancellous bone primarily consists of areas of medullary bone, which is porous in nature, as opposed to cortical bone like that of the femoral shaft, which has thick structural walls within which fixation can easily be secured. These pelvic ring injuries can be fixed with internal or external fixation methods, or a combination. The most widely used internal fixation construct consists of a cannulated screw with a washer that are both placed over a guidewire. External fixation consists of pins that are placed into bone and are secured to bars outside the skin, or to a bar placed in a subcutaneous fashion at the anterior pelvis. Both of these described internal and external fixation methods are performed percutaneously, through small minimally-invasive incisions, and would be indicated for fractures that are minimally-displaced or able to be reduced in an indirect fashion. A widely displaced fracture of the pelvis would require open reduction and internal fixation, which means large incisions through which plates and screws could be affixed to the bone after the fractures are directly reduced using clamps or other devices. The invention can also be used in these cases with open reduction and internal fixation to secure the posterior aspect of the pelvic ring. The sacroiliac locking fixation bolt can be used alone or in conjunction with percutaneous or open surgery of the anterior pelvic ring.

The issue that occurs with current fixation hardware is loosening or loss of fixation, which leads to continued instability of the pelvis and a patient's inability to mobilize or ambulate, and possibly to a revision surgery. The multi-axial forces of ambulation and mobilization applied to the fixation devices cause loosening that traditional fasteners are not able to withstand. Improved implant design that allows for reinforcement of fixation would be desirable for optimizing patient healing and outcomes following these devastating injuries. The invention in this embodiment consists of metal implants that lock to each other and provide a compressive force across the posterior pelvis, including a screw and a locking bolt that each have an abutment washer against a monolithic head, and that affixes within a safe transiliac transsacral corridor.

### DETAILED DESCRIPTION OF THE DRAWINGS

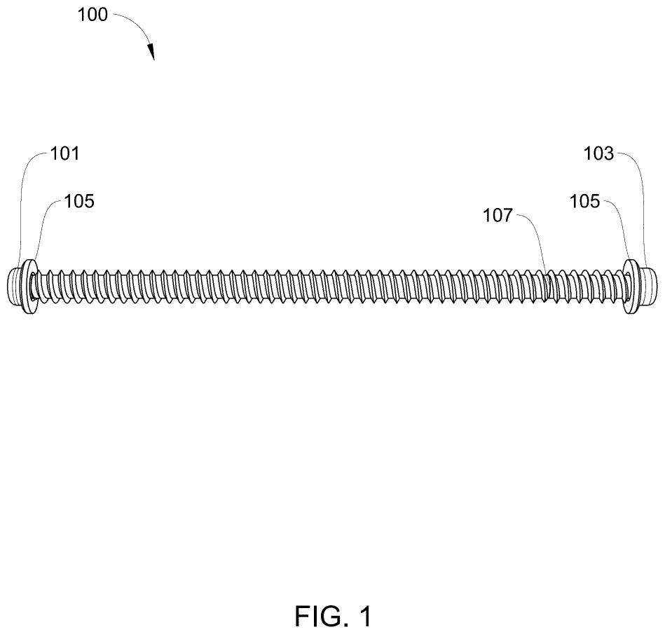

Generally referring to FIGS. 1-7, sacral fixation is constructed in accordance with an embodiment that includes one or more bone fixation implants configured to be fixed to first and second bony locations in a patient's body. Posterior sacroiliac fixation is currently typically performed using a screw that can accommodate a washer to distribute forces from the screw head to the lateral cortex of the ilium on the side nearest to the surgeon, which would be referred to as the near cortex. If that screw is affixed from the near cortex of the ilium through the sacrum to the far cortex of the second ilium, respective to the surgeon, then this embodiment would be configured to be fixed in a transiliac transsacral fashion through both iliac bones and the sacrum. FIG. 1 depicts an improvement to the current available implants as it would be fully assembled, implant construct as a whole is 100 in a perspective view. The screw has an elongate body that has helical threads about its outer surface that have a major diameter, a minor diameter, and a pitch. 101 is the screw head, 105 is a washer, and an identical washer is on either end of the construct, with the bolt head labeled 103. The interface between the screw and the locking bolt is marked 107 and shows where the two components come together. The washers 105 are abutment surfaces that increase the contact area between the implant and the bone at the lateral surfaces of the ilium, and allow for compression to reduce and allow healing of the pelvic injury while also preventing migration of the screw or locking bolt into the ilium on either side. The bolt 107 has a shorter body than that of the screw, but likewise has helical threads about its outer surface that have a major diameter, a minor diameter, and a pitch that matches that of the long counterpart.

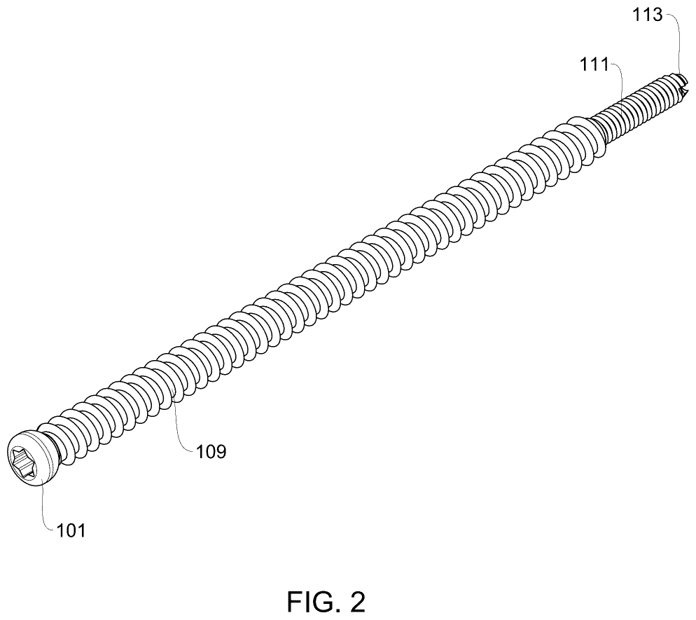

In FIG. 2, a perspective view of the screw is shown. The helical threads about the elongate shaft body have different sectional moduli and are shown as 109, a cancellous pitch, as is current standard for posterior pelvic fixation, and a locking pitch labeled 111 at its distal end. The thread pitch of the majority of the shaft 109 is larger than that of the distal tip 111. This differential pitch allows the locking bolt of the second part of the embodiment to be able to affix to the elongate body of the screw. The tip of the screw is self-drilling and self-tapping, and that end is marked 113. The self-drilling tip of the screw 113 is designed to bore a hole into the bone of the ilium and sacrum.

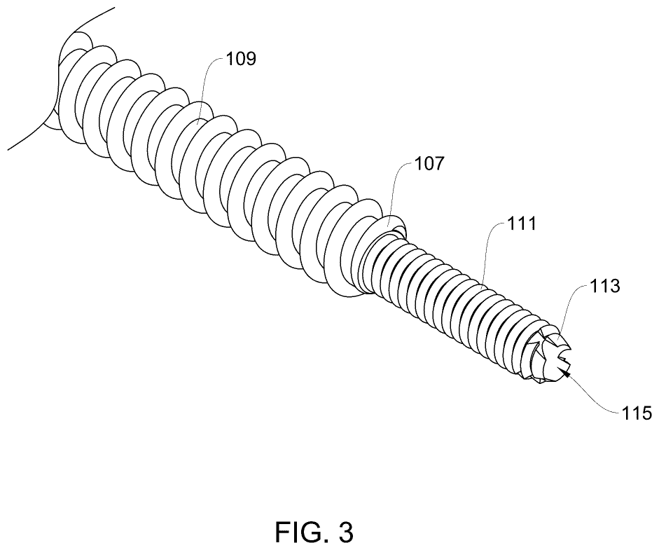

FIG. 3 is an enlarged perspective view of the tip of the screw showing the variation in pitch in more detail. The self-drilling self-tapping end of the screw 113 is better visible, as well as the cannulation 115 within the screw itself. The cannulation 115 is a channel through which a guide wire passes, and the implant 100 rotates around said guide wire. The cancellous threads also have a self-tapping component at the interface where the two variable pitch threads meet, 107.

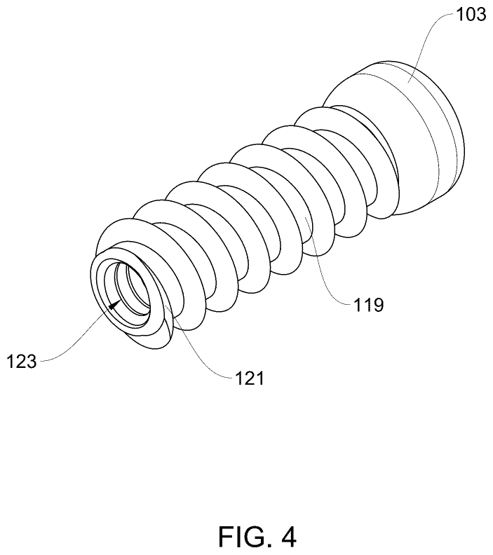

FIG. 4 depicts an enlarged perspective view of the locking bolt portion of the fixation construct, with 123 showing the inner threads that match the pitch of the outer threads of the distal end of the screw 111. These matching threads allow the bolt to mate with the screw, providing a locked construct. This bolt has a cannulation that fits over a guide wire, as does the screw. 103 shows the head of the bolt, which has the same dimensions as the head of the screw 101, and the shaft 119 with the same cancellous pitch as that of the screw 109 (FIG. 2). 121 shows the self-tapping threads of the tip of the bolt, and these allow the implant to cut its own path through the bone of the pelvis. This bolt provides an embodiment of the sacral fixation construct that affixes to a second bony location than the first, the far cortex of the ilium. This part of the construct is inserted within the body by use of the guide wire, that fits through the channel in the head of the implant.

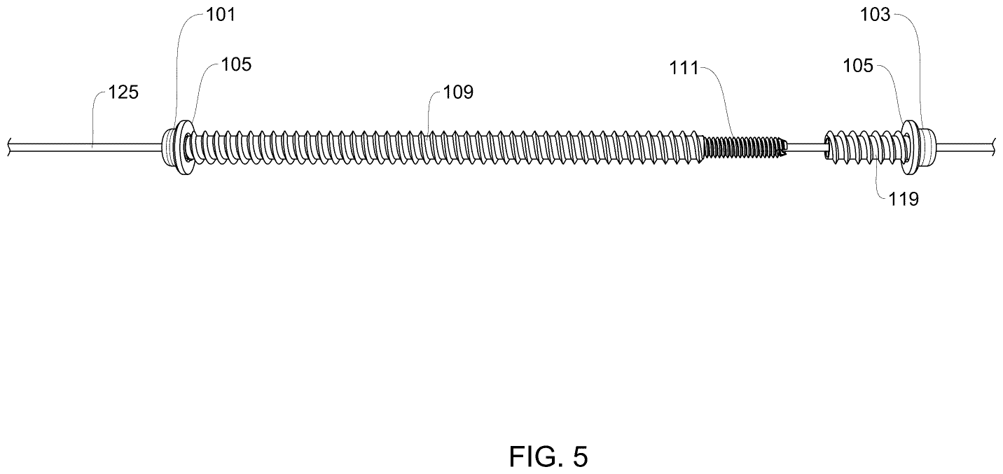

FIG. 5 is an exploded perspective view showing the various parts of the fixation construct over a guidewire 125 that fits through the cannulation channel of the screw and the bolt. The outer threads of the distal end of the screw 111 match the inner threads of the bolt 123 (FIG. 4), which allows the pieces to lock together and provides a stable fixation construct for the posterior pelvis; this is an improvement to prior art and provides mating implant components. Unlike previous implant structures, the locking bolt of this construct has outer threads 119 that cut into and hold within the distal ilium bone, providing stability and preventing implant cutout and failure.

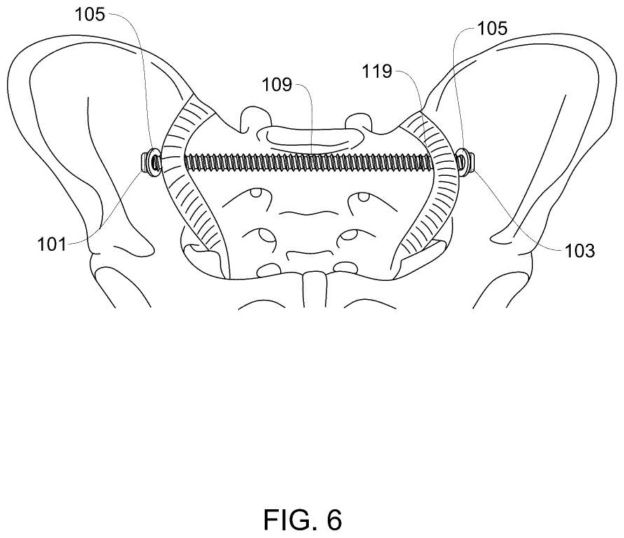

FIG. 6 depicts a human bony pelvis with a sacroiliac fixation locking bolt in place, assembled, in the upper sacral segment S1. This is a bony corridor within which safe fixation can be placed when the anatomy allows. In cases of sacral dysmorphism, which is an aberrant but relatively common anatomical difference, where the physical characteristics of the upper sacral segment (S1) would disallow transiliac transsacral fixation, the second sacral segment (S2) would typically be able to accommodate the transiliac transsacral implant while an iliosacral implant would be used in S1. The screw head 101 abuts a washer 105, which distributes the force more evenly at the lateral cortex of the near-side ilium. The bolt head 103 abuts a washer 105, which provides the same force distribution on the far-side ilium, which is the second bony fixation location. The shaft of the screw 109 and of the bolt 119 have the same pitch, and this provides bony purchase within the less dense bone of the medullary canals of the sacrum and the ilium, and also stable fixation within the cortices of the ilia and sacrum.

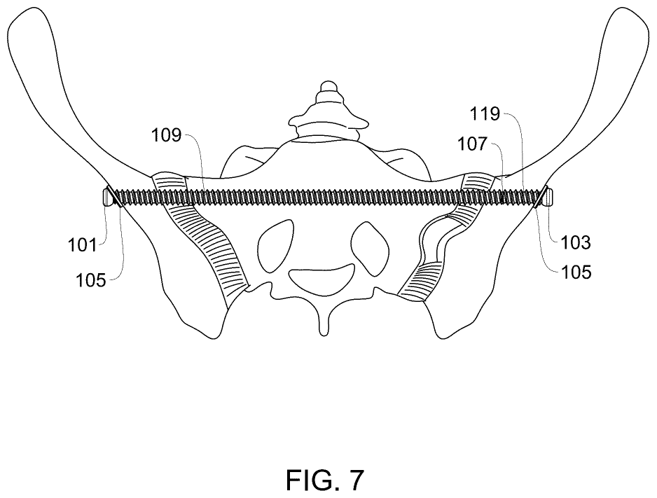

FIG. 7 provides a cross-sectional view of the posterior pelvic ring, with the sacroiliac fixation locking bolt within the safe bony corridor of the pelvis. The interface 107 where the screw and bolt connect is more clearly seen. The washers 105 on either side of the construct are depicted as they would be in situ, flush against the lateral iliac cortices. The heads of the screw 101 and bolt 103 are identical, as are the pitches of the screw 109 and bolt 119. This pitch 109 and 119 allows for stable fixation within the bone of the pelvis.

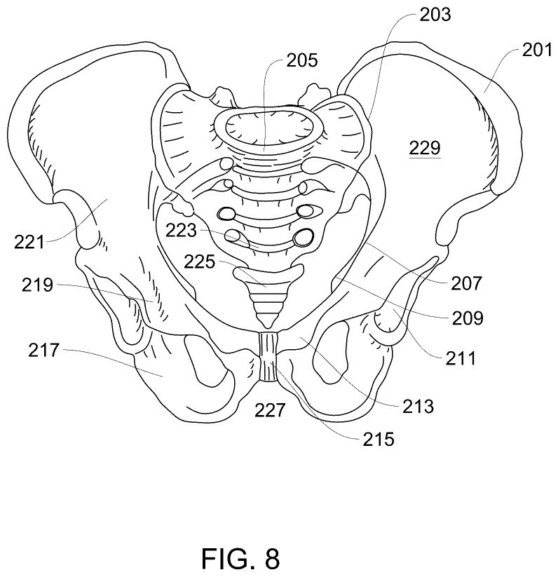

FIG. 8 depicts a human bony pelvis 227 with the pertinent structures labeled. The sacrum 205 is the central portion of the posterior bony pelvis and the distal aspect of the axial skeleton, comprising sacral vertebrae S1 through S5. The cranial border of the upper sacrum, is the anterior sacral promontory, and this provides a radiographic landmark during fluoroscopic visualization intra-operatively. 203 shows the sacroiliac joint between the sacrum 223 and the ilium 229. The sacroiliac joint can be injured by tearing of the ligaments anteriorly, posteriorly, or both, or can be compromised by way of a sacral or iliac fracture on either side of the joint. The coccyx 225 is at the caudal aspect of the triangular sacrum and is the terminal vertebra of the spinal column. The iliac crest 201 is the cortical superior edge of the iliac bone, and is a palpable bony landmark of the pelvis. 221 is the iliac fossa where the iliacus muscle resides, and this is maximally visualized on an oblique view radiographically and fluoroscopically. The pelvic brim 207 is the border of the anterior column of the acetabulum, and 219 is the pubic root where the pubis meets the ilium. 217 is the ischium, the posterior portion of the anterior pelvic ring and the structure upon which the body weight is supported while a person is in the seated position. The pubic symphysis 215 is the fibrocartilaginous structure at the anteriormost aspect of the pelvis. A rupture of the symphysis pubis can be reduced indirectly by fixing the posterior pelvis, as would be possible using the posterior sacroiliac fixation device as recommended in this literature. The pubic tubercle 213 is the insertion point for the rectus abdominus and is a bony prominence used for open and percutaneous fixation surgery of the pelvic ring. The ischial spine 209 is a point at which multiple ligamentous structures attach to form the pelvic bowl, and a radiographic landmark. The hips are seated within the acetabula bilaterally, 211, and this articulation provides the point of contact between the lower appendicular skeleton and the pelvis, which connects the lower limbs to the axial skeleton.

Referring generally to FIGS. 5 through 8, the surgical method for insertion of this implant is minimally-invasive and comprises small percutaneous incisions on the posterior aspect of the hips, using radiography in the form of fluoroscopic imaging to target safe defined trajectories through bony corridors of the pelvis. A guide wire is used as the axis about which the embodiments are inserted to maintain safe trajectory through one iliac bone 229, the sacrum 205, and the second iliac bone. The locking bolt 119 is likewise inserted over the guide wire 125, which is to be placed through the bone and into the soft tissue of the contralateral hip, out the skin of the other side. A small incision can then be made over the wire, allowing for obturating instruments to be used sequentially, followed by the bolt 119 over the wire. The bolt can then be threaded onto the elongate body of the screw 111 to complete the posterior sacroiliac fixation bolt construct, holding purchase in both iliac bones and the sacrum.

As the elongate body of the screw is placed with the abutment washer flush against the lateral cortex of the first ilium, it becomes secure within the posterior pelvis. Then, as the locking bolt is threaded into the second ilium and its internal threads 123 mate with the external threads of the distal tip of the elongate screw body 111, a compressive force is applied to the posterior pelvis. This compression promotes bone healing by reducing the fracture or widened sacroiliac joint(s) as the bony segments are drawn closer together, to a more anatomic relationship than was present in the alignment post-injury.

### CROSS-REFERENCE TO RELATED APPLICATIONS

Not applicable

1. An implantable sacral fixation implant consisting of
a. a screw segment with a proximal and distal end of different pitches,
b. said screw shaft with a contained channel that accommodates a guidewire,
c. said screw has a monolithic head and separate washer that are designed to abut the lateral ilium and prevent further intrusion of the implant into the bone and also provide compression,
d. and a locking member that unites with the screw segment.

2. The fixation construct is completed with a locking bolt that secures itself to the sacral fixation implant as recited in claim 1, acting as a locking cap to the sacral fixation implant.

3. The sacral fixation implant as recited in claim 1, wherein the channel further defines the aperture accommodates a guidewire.

4. The sacral fixation implant as recited in claim 1, wherein the majority of the shaft has a first pitch and the distal segment of the shaft has a second pitch that is smaller than the first.

5. The sacral fixation implant as recited in claim 1, wherein the width of the shaft at the proximal end is greater than the width of the shaft at the distal end.

6. The sacral fixation implant as recited in claim 1, wherein the distal tip has a self-cutting end, which is configured to bore a hole through the bone over the guidewire as recited in claim 3.

7. The sacral fixation implant as recited in claim 1, wherein the distal aspects of both the smaller and larger threads have a self-tapping component that cuts the thread pitch within the bone.

8. The sacral fixation implant as recited in claim 1 wherein the shaft defines at least one external thread at the proximal end to be driven into the ilium bone.

9. The sacral fixation implant as recited in claim 1 wherein the guidewire is configured to be driven into the contralateral ilium extending through the implant segment and out the other side, penetrating the skin of the distal side.

10. The locking bolt as recited in claim 2 with a monolithic head and separate washer that are designed to abut the lateral ilium and prevent further intrusion of the implant into the bone and also provide compression.

11. The locking bolt as recited in claim 2 has inner threads that match the outer threads of the distal portion of the screw member as recited in claim 4 and these are configured to fasten to each other when fully seated. This allows the bolt as recited in claim 2 to lock to the screw as recited in claim 1 that is placed from the proximal to distal aspect of the pelvis, as referenced from the operative side at which the surgeon is standing.

12. The locking bolt as recited in claim 2 with a contained channel that accommodates a guidewire.

13. The locking bolt as recited in claim 2 has outer threads along its shaft that match the pitch of the sacral fixation implant as recited in claim 4.

14. The outer threads of the locking cap as recited in claim 12 are configured to be at least one thread driven at least into the ilium bone.

15. The sacral fixation implant as recited in claim 1 wherein the implant segment is designed to rotate around the guidewire as recited in claim 3.

16. The locking bolt as recited in claim 2 wherein the implant segment is designed to rotate around the guidewire as recited in claim 3, and thread onto the sacral fixation implant as recited in claim 1.

17. The sacral fixation implant as recited in claim 1 and the locking bolt as recited in claim 2 wherein the hardware is enclosed within the iliac and sacral bones within a safe bony corridor and the abutment areas and separate washers are external to the bone of the pelvis.
